(19) United States
(12) Patent Application Publication
(10) Pub. No.: US 2026/0252866 A1
(43) Pub. Date: Aug. 27, 2026

(54) MANUFACTURING METHOD OF BRAIN NEURAL NETWORK MODEL, AND BRAIN NEURAL NETWORK MODEL USING THE SAME

(71) Applicant: POSTECH Research and Business Development Foundation, Pohang-si (KR)

(72) Inventors: Mihyeon BAE, Pohang-si (KR); Joeng Ju Kim, Pohang-si (KR); Jinah Jang, Pohang-si (KR); Dong-Woo Cho, Pohang-si (KR)

(73) Assignee: POSTECH Research and Business Development Foundation, Pohang-si (KR)

(21) Appl. No.: 19/350,450

(22) Filed: Oct. 6, 2025

(30) Foreign Application Priority Data
Feb. 26, 2025 (KR) 10-2025-0025055

(51) Int. Cl. G06N 3/06

(52) U.S. Cl. CPC G06N 3/061

(57) ABSTRACT

The present invention relates to a manufacturing method of a brain neural network and a brain neural network model manufactured thereby. It is characterized by implementing compartmentalized brain structures using 3D cell printing with brain-specific bioink and sacrificial material. This enables the effective separation of axons and cell bodies without physical barriers, thereby achieving the effect of effectively mimicking the brain's intrinsic structure and synaptic dynamics.

### CROSS-REFERENCE TO RELATED APPLICATION

This application claims the benefit of Korean Patent Application No. 10-2025-0025055, filed on Feb. 26, 2025, the entire contents of which are hereby incorporated by reference in their entirety.

### TECHNICAL FIELDS

The teachings in accordance with exemplary and non-limiting embodiments of the present invention relate generally to a brain neural network model, particularly to a manufacturing method of brain neural network model capable of similarly reproducing a compartmentalized brain structure, and to a brain neural network model using the same.

### BACKGROUND ARTS

The brain is a vital organ that coordinates cognitive and motor functions while maintaining homeostasis, partitioned into grey matter and white matter. Grey matter consists of neuronal cell bodies, whilst white matter comprises axonal bundles, enhancing the speed and efficiency of neural signal transmission. In particular, the unidirectional axonal networks within white matter transmit signals efficiently, much like electrical circuits and damage to these networks may lead to pathological phenomena such as neurodegenerative diseases. Neurodegenerative diseases cause dysfunction in the axonal network, neuronal cell death, and a reduction in brain volume. However, as the mechanisms underlying their onset are not yet fully understood, in vitro models that mimic the physiological structure of the brain are required for research purposes.

Currently developed 2D and 3D models have limitations in that they cannot perfectly reproduce neural signal transmission and axonal dynamics. 2D models implement neural networks on a flat plane, thus failing to realize the three-dimensional structure of complex neural networks. Consequently, they simplify neural connectivity and cannot replicate the complex 3D synaptic architecture of the brain. 3D brain organoids are useful for simulating brain development through self-assembling cells and for studying various neurological disorders. However, they suffer from limitations including the random connectivity of axonal networks, difficulties in controlling size and shape, and constraints in post-processing for optical observation. These limitations reduce the consistency of experimental results, posing challenges for studying the pathological mechanisms of neurodegeneration.

To address this issue, 3D bioprinting and brain-specific bioink fabrication techniques are emerging as promising alternatives. Utilizing 3D bioprinting and decellularized bioinks that mimic the brain microenvironment enables the precise engineering of neural tissue, allowing the implementation of anatomical features such as the blood-brain barrier (BBB) and axonal bundles.

However, most current research aiming to mimic brain neural networks ex vivo remains at a fundamental level, attempting to compartmentalize the brain's constituent parts. It has primarily focused on co-culturing a few types of brain-forming cells in simple microfluidic models or creating spheroids for application to neurodegenerative diseases such as Alzheimer's. Furthermore, attempts to regulate axonal direction and neuronal maturity using electrical stimulation exist, but these remain at a very rudimentary level.

Current 3D bioprinting technology has limitations in controlling axonal growth direction. To overcome this, alongside implementing neural tissue via 3D bioprinting, biological stimuli such as electrical stimulation are required. Electrical stimulation can non-invasively control the direction of the axonal growth cone, significantly enhancing the functionality and applicability of 3D bioprinting models.

### PRIOR TECHNICAL DOCUMENT

### Patent Document

PCT International Patent No. WO 2023-201047 A1 (Oct. 19, 2023)

### SUMMARY OF INVENTION

### Technical Subject

The present invention aims to address the aforementioned issues by providing a brain neural network model capable of effectively simulating the brain's intrinsic structure and synaptic dynamics.

### Technical Solution

The manufacturing method of brain neural network model according to an exemplary embodiment of the present invention for achieving the aforementioned purpose may comprise the steps of: printing a first bioink containing neural progenitor cells (NPCs) within a frame having a predetermined shape; printing a sacrificial material onto a portion of the printed first bioink; removing the printed sacrificial material; printing a second bioink, which is devoid of cells, onto the area where the sacrificial material has been removed; and maturing the printed first bioink and second bioink.

A brain neural network model according to another embodiment of the present invention may comprise: a first region formed by printing a first bioink containing neural progenitor cells (NPCs) manufactured by the manufacturing method; and a second region formed by printing a second bioink without cells onto a portion of the printed first bioink.

A brain neural network model according to still another embodiment of the present invention may comprise: a first region formed by printing a first bioink containing neural progenitor cells (NPCs) within a frame having a predetermined shape; and a second region formed by printing a cell-free second bioink onto a portion of the printed first bioink after printing and removing a sacrificial material; wherein the neural progenitor cells (NPCs) in the first region differentiate into neurons, and axons extend into the second region,

A brain neural network model according to still another embodiment of the present invention may comprise: a first region formed by printing a first bioink containing neural progenitor cells (NPCs) within a frame having a predetermined shape; a second region formed by printing a cell-free second bioink after printing and removing a sacrificial material on a portion of the printed first bioink; and an electrode for applying an electric field to the first and second regions; wherein the neural progenitor cells (NPCs) in the first region differentiate into nerve cells, and axons extend into the second region,

A brain neural network model according to still another embodiment of the present invention may comprise: a first region formed by printing a first bioink containing neural progenitor cells (NPCs) within a frame having a predetermined shape; a second region formed by printing a cell-free second bioink after printing and removing a sacrificial material on a portion of the printed first bioink; a third region formed by printing a cell-free third bioink after printing and removing a sacrificial material on a portion of the printed second bioink, wherein the second region and the third region have an intersecting point, and the neural progenitor cells (NPCs) in the first region differentiate into nerve cells, and axons extend into one or more regions out of the second region and third region,

Other specific matters of the exemplary embodiments are included in the detailed description and drawings.

### Advantageous Effects

The present invention is characterized by the implementation of compartmentalized brain structures using 3D cell printing with brain-specific bioink and sacrificial materials. This enables the effective simulation of the brain's intrinsic architecture and synaptic dynamics by separating axons and cell bodies without physical barriers.

Moreover, the present invention enables precise control of axon-cell body compartmentalization by accurately positioning cells and adjusting distances within neural tissue via 3D bioprinting.

Furthermore, the present invention is characterized by employing electrical stimulation to control axon orientation after fabricating the compartmentalized structure of the brain neural network model. This invasive control technique utilizing electrical stimulation can regulate not only axon direction but also neuronal maturity.

### BRIEF DESCRIPTIONS OF THE DRAWINGS

FIG. 1 is a schematic diagram illustrating the manufacturing method of a brain neural network model according to an exemplary embodiment of the present invention.

FIG. 2 is an overview diagram (A) of the brain neural network model production using 3D bioprinting according to an embodiment of the present invention and an actual production photograph (B) of the brain neural network model.

FIG. 3 presents evaluation results from the brain neural network model according to an embodiment of the present invention, showing compartmentalization formation based on the width of the acellular region and the promotion of axonal extension through BDNF treatment: (A) optimization of the acellular region printing width to induce axonal elongation, (B) Fluorescent image of the compartmentalized area after three weeks of maturation, (C) Immunofluorescent image of the BDNF-treated compartmentalized model, and (D) RT-PCR results for neuronal/glial cell differentiation).

FIG. 4 shows the brain neural network model incorporating electrodes according to an embodiment of the present invention, and the results of electrical stimulation experiments using it: (A) the process of implementing electrical stimulation in the brain neural network model, (B) the acrylic housing for electrical stimulation, (C) live and dead analysis after electrical stimulation, (D) schematic of calcium ion distribution within neurons under DC and AC electric fields, (E) immunofluorescence image after 14 days to confirm the growth direction of NPCs induced by electric fields under various voltage conditions, (F) analysis of axonal growth pathways for neuronal growth direction, (G) quantitative analysis of axonal growth angles under various electrical stimulation conditions, (H) confirmation of NPC growth length induced by electric fields under various frequency conditions over 14 days via immunofluorescence, (I) quantitative quantification of NPC growth length).

FIG. 5 shows the brain neural network model comprising electrodes and a cross-shaped channel according to an embodiment of the present invention, and the results of electrical stimulation experiments using it. ((A) schematic for confirming axonal growth direction under electric field induction (B) immunofluorescence tile scan for confirming axonal network formation (C) quantitative analysis of synaptophysin (D) quantitative analysis of calcium signals for spontaneous neural activity in the brain neural network).

FIG. 6 shows the results of applying the brain neural network model according to an embodiment of the present invention to an alcohol-induced neurodegeneration model. (A) schematic diagram illustrating the application of the brain neural network model to an EtOH-induced neurodegeneration model (B) acute calcium signaling dysfunction under EtOH treatment (C) axonal deformation due to chronic alcohol consumption (D) immunofluorescence results for amyloid-β (E) immunofluorescence results for t-tau (F) immunofluorescence results for p-tau (G) quantitative analysis of immunofluorescence intensity for amyloid-β, (H) t-tau, (I) p-tau).

### DETAILED DESCRIPTIONS OF THE INVENTION

The present invention may have various modifications and have various exemplary embodiments, and specific embodiments will be illustrated in the drawings and described in detail in the detailed description. However, it is not intended to limit the present invention to specific embodiments, and should be understood to include all transformations, equivalents, or substitutes included in the spirit and technical scope of the present invention. In describing the present invention, when it is determined that a detailed description of a related known technology may obscure the gist of the present invention, the detailed description thereof will be omitted. The terms used in the present application are intended to explain the specific embodiments and are not intended to limit the invention. The singular form may include the plural form unless otherwise specified in the text.

In this application, the terms “include” or “have” are intended to specify the existence of features, numbers, steps, actions, components, components, or a combination thereof described in the specification, and should be understood not to preclude the existence or addition of one or more other features, numbers, steps, actions, components, components, or combinations thereof.

Terms such as first and second may be used to describe various components, but the components should not be limited by the above terms. The above terms are used only for the purpose of distinguishing one component from another component.

Throughout this specification, the symbol ‘%’ used to denote the concentration of a particular substance shall, unless otherwise stated, represent (weight/weight) % for solid/solid, (weight/volume) % for solid/liquid, and (volume/volume) % for liquid/liquid.

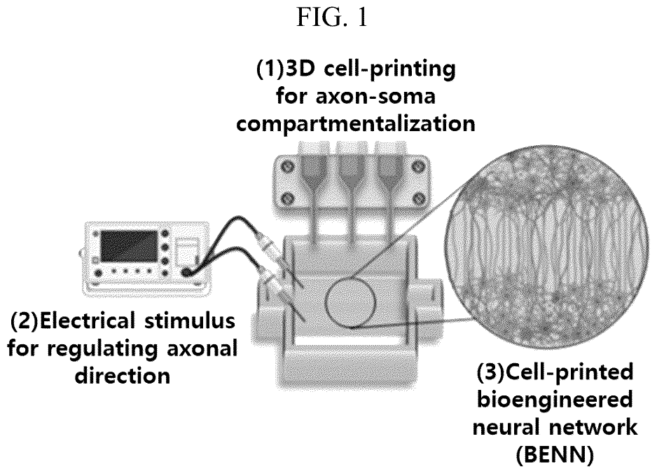

FIG. 1 is a schematic diagram illustrating a manufacturing method of a brain neural network model according to an exemplary embodiment of the present invention.

One of the brain's most significant characteristics is its compartmentalized structure, divided into grey matter (areas containing cells) and white matter (areas without cells). To realize this, the inventors established a compartmentalized design using 3D bioprinting technology and a hybrid hydrogel containing decellularized extracellular matrix (dECM) and Matrigel, which separates the neuron's soma and axon. The aforementioned hybrid hydrogel can promote the maturation of neural cells and aid their activation.

Furthermore, the inventors developed a brain neural network model (Bioengineered Neural Network, BENN) combining 3D bioprinting and electrical stimulation. In this model, brain-specific decellularized bioink and sacrificial material (Pluronic F-127, PF-127) printing compartmentalized axons and somas to mimic grey and white matter. Periodic electrical stimulation was utilized to form a unidirectionally aligned axonal network without physical barriers. BENN demonstrated the ability to accurately mimic neural signal transmission while reproducing brain modeling characteristics such as spontaneous calcium responses. Furthermore, to validate BENN's applicability, an alcohol-induced neurodegenerative model was implemented by controlling alcohol concentration. BENN successfully visualized axonal deformation across varying alcohol concentrations, proving itself a useful tool for studying the pathological mechanisms of neurodegeneration.

Accordingly, the present invention is a brain neural network model that overcomes the limitations of existing models and precisely mimics the physiological structure of the brain by integrating 3D bioprinting with electrical stimulation. This model serves as a crucial tool for neural signal transmission, analysis of pathological factors through research into the mechanisms of neurodegenerative diseases, and drug development, and can be utilized across various fields of neuroscience research.

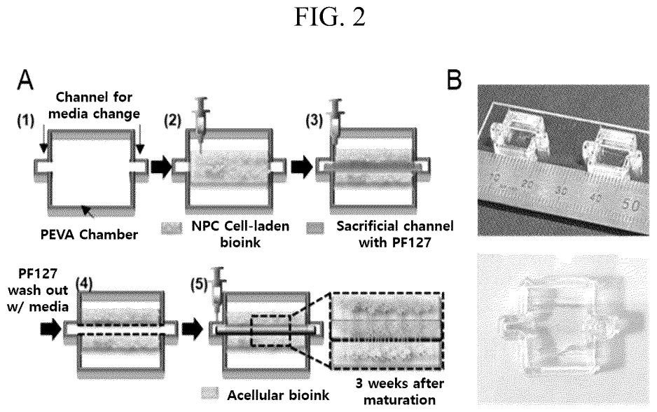

FIG. 2 shows an overview diagram (A) of the brain neural network model fabrication using 3D bioprinting according to one embodiment of the present invention, and an actual fabrication photograph (B) of the brain neural network model.

One embodiment of the present invention is a manufacturing method of a brain neural network model, comprising the steps of: printing a first bioink; printing a sacrificial material; removing the sacrificial material; printing a second bioink; and maturing the bioink.

The step of printing the first bioink comprises printing the first bioink, which contains neural progenitor cells (NPCs), within a frame having a predetermined shape. The first bioink contains NPCs, and when printed, it can form a first region (grey matter region) containing cells. As an example of the present invention, the first bioink may comprise neural progenitor cells (NPCs), brain decellularized extracellular matrix (BdECM), and Matrigel.

The frame serves as the support structure for the model according to the present invention, and its shape is not specifically limited. As an example of the present invention, the frame may have a shape comprising a bottom portion, a side perimeter portion, and an upper opening.

The method of printing the first bioink within the frame is also not specifically limited. For example, it is possible to print by moving a 3D printing nozzle containing the first bioink through the opening at the top of the frame while uniformly discharging the first bioink onto the bottom surface inside the frame.

The step of printing the sacrificial material comprises printing the sacrificial material onto a portion of the printed first bioink. The sacrificial material is not specifically limited and any material known in the art may be utilised. The present invention is for producing a cell-free second region (white matter region) by removing part of the first bioink using the sacrificial material and then printing the second bioink described below onto this area. The sacrificial material may be printed across portions of the region where the first bioink is formed, for example, across one side or the central region of the first bioink.

As an example of the present invention, printing the sacrificial material may be performed transversely across the longitudinal direction of the printed first bioink. In this case, a model can be produced wherein the first region (grey matter region) formed by the first bioink is positioned on both sides of the second region (white matter region) formed by the second bioink.

In one embodiment of the present invention, printing the sacrificial material may involve discharging the sacrificial material into the printed first bioink while the nozzle discharging the sacrificial material is in a locked state. In this case, the sacrificial material can be introduced into the interior of the first bio-ink, enabling more complete removal of the first bio-ink and effectively partitioning the first and second regions.

The step of removing the sacrificial material involves removing the printed sacrificial material. The method for removing the sacrificial material is likewise not specifically restricted, and various methods known in the art may be employed. For example, it is possible to remove it by injecting medium only into the printed sacrificial material.

As an example of the present invention, removing the sacrificial material involves introducing the medium into the interior of the frame and leaving it to stand, thereby enabling the sacrificial material to be removed. An advantage of uniformly introducing cell culture medium throughout the frame is that it allows the NPC contained within the first bioink to be cultured whilst simultaneously removing the sacrificial material.

The step of printing the second bioink involves printing the second bioink, which is devoid of cells, onto the area where the sacrificial material has been removed. The second bioink does not contain cells and, in the model according to the present invention, forms a second cell-free area (the white matter area). As an example of the present invention, the second bioink may comprise brain decellularized extracellular matrix (BdECM) and Matrigel. The method for printing the second bioink is likewise not specifically limited.

The step of maturing the bioink involves maturing the printed first bioink and second bioink. In this specification, “maturing” refers to the process of differentiating the NPCs contained within the first bioink into neural cells or allowing axons to propagate from them. Therefore, the method of maturing is not specifically restricted. It is also possible to simply leave it undisturbed for a predetermined period.

As an example of the present invention, the maturation may involve introducing a culture medium into the frame containing the printed first bioink and second bioink, and culturing the neural progenitor cells (NPCs) of the first bioink. This can further promote the differentiation and axonal outgrowth of the NPCs.

In one embodiment of the present invention, the maturation step may involve introducing a medium containing brain-derived neurotrophic factor (BDNF) into the frame containing the printed first bioink and second bioink, and culturing the neural progenitor cells (NPCs) from the first bioink for a period ranging from two to four weeks. BDNF can further promote the differentiation and maturation of neurons, as well as the differentiation of glial cells, and a culture period of approximately three weeks is most effective.

As an example of the present invention, the medium containing brain-derived neurotrophic factor (BDNF) may be a medium in which said brain-derived neurotrophic factor (BDNF) is included at a concentration of 5 ng/ml to 15 ng/ml. As can be confirmed in the experimental examples described below, the inventors sought to identify the optimal concentration at which BDNF is included and confirmed that effective separation of axons and cell bodies is most suitable within the aforementioned concentration range.

This invention is characterized by implementing compartmentalized brain structures using 3D cell printing with brain-specific bioink and sacrificial materials. This enables the separation of axons and cell bodies without physical barriers, thereby effectively mimicking the brain's intrinsic structure and synaptic dynamics.

A brain neural network model according to another embodiment of the present invention comprises: a first region (grey matter region) formed by printing a first bioink containing neural progenitor cells (NPCs), manufactured by the aforementioned manufacturing method; and a second region (white matter region) formed by printing a second bioink, devoid of cells, onto a portion of the printed first bioink.

Existing channel-based microfluidic models rely on simplified physical barriers, which limit the formation of natural synaptic dynamics, including synaptic pruning and neurotransmitter release. However, the barrier-free compartmentalization of the model according to the present invention enables the observation of natural synaptic network formation and prevents antibody dyes or proteins from unnecessarily attaching to the barrier walls.

A brain neural network model according to still another embodiment of the present invention comprises: a first region formed by printing a first bioink containing neural progenitor cells (NPCs) within a frame of predetermined shape; and a second region, formed by printing a cell-free second bioink onto an area where a sacrificial material was printed onto and subsequently removed from a portion of the printed first bioink; wherein the neural progenitor cells (NPCs) in the first region differentiate into neurons, and axons extend into the second region.

As an example of the present invention, the second region may be formed in the longitudinal direction of the first region. The second region may be formed at various positions on a portion of the first region, irrespective of size or direction. When the second region is formed in the longitudinal direction of the first region, a model with a consistent structure in a specific direction may be formed. The second region may also be formed midway across the first region in the longitudinal direction of the first region.

In one embodiment of the present invention, the second region may have a width within the range of 100 μm to 300 μm. As shown in the experimental examples described below, the inventors evaluated the compartmentalization-forming ability by varying the width of the cell-free second region. The results confirmed that a width of approximately 200 μm is most favorable for axonal pathway formation, enabling inter-regional connectivity while preventing the integration of neuronal cell groups. That is, if the width is below the aforementioned range, there is a problem where separated grey matter regions (cellular regions) re-merge, making it difficult to generate a compartmentalized structure. Conversely, if the width exceeds the aforementioned range, there is the disadvantage that axonal extension from the first region to the second region becomes difficult. This invention enables precise control of axon-cell body compartmentalization by accurately positioning cells and adjusting distances within neural tissue via 3D bioprinting.

The present invention enables precise control of axon-cell body compartmentalization by accurately positioning cells and adjusting their distances within neural tissue via 3D bioprinting. This demonstrates the potential of this approach as a precise and controllable technology for regulating interactions and functions within neural networks. Furthermore, as the engineered neural structure maintains a thickness of approximately 300 μm, real-time optical and immunofluorescence analysis is possible without post-processing steps such as sample clearing or sectioning. Eliminating these post-processing steps ensures more accurate preservation of expressed pathophysiological phenotypes, and real-time analysis enables the identification of degeneration points within the in vitro model.

As an example of the present invention, it is possible to further include within the frame a medium containing brain-derived neurotrophic factor (BDNF) at a concentration of 5 ng/ml to 15 ng/ml. As described above, the inventors sought to identify the optimal concentration at which BDNF is included and confirmed that effective separation of axons and cell bodies is most suitable within the aforementioned concentration range.

A brain neural network model according to still another embodiment of the present invention comprises: a first region formed by printing a first bioink containing neural progenitor cells (NPCs) within a frame having a predetermined shape; a second region formed by printing a cell-free second bioink after printing and removing a sacrificial material on a portion of the printed first bioink; and electrodes for applying an electric field to the first and second regions; wherein the neural progenitor cells (NPCs) in the first region differentiate into neurons, and axons extend into the second region.

The number or position of said electrodes may not be specifically limited. This invention is characterized by using electrical stimulation to control axonal direction after fabricating the compartmentalized structure of the brain neural network model. This invasive control technique utilizing electrical stimulation can regulate not only axonal direction but also neuronal maturity. The provision of electric or magnetic fields by electrodes according to the present invention can be applied not only to in vitro cell culture but also to the regeneration of neural tissue damaged by neurological disorders. This invention enables more accurate and dynamic study of neural behavior and facilitates in-depth research into neural signal transmission and the development of neurological disorders.

As an example of the present invention, the second region may be formed in the longitudinal direction of the first region, and the electrode may be positioned perpendicular to the second region. When the electrode is positioned perpendicular to the longitudinal direction of the second region or the first region, the electric or magnetic field generated by said electrode is applied along said longitudinal direction. Axons then grow in a direction perpendicular to said electric or magnetic field (the direction connecting the first and second regions), enabling more similar and effective connections between grey matter and white matter.

A brain neural network model according to still another embodiment of the present invention comprises: a first region formed by printing a first bioink containing neural progenitor cells (NPCs) within a frame having a predetermined shape; a second region formed by printing a cell-free second bioink onto an area where a sacrificial material was printed and subsequently removed from a portion of the printed first bioink; and a third region formed by printing a cell-free third bioink onto an area where a sacrificial material was printed and subsequently removed from a portion of the printed first bioink and second bioink; wherein the second region and the third region have intersecting points, and the neural progenitor cells (NPCs) in the first region differentiate into neural cells, with axons extending into one or more of the second and third regions.

The brain neural network model according to the present invention is characterized by being formed such that the second and third regions, which are devoid of cells, intersect at a point, thereby possessing a cross-shaped channel. Thus, fabricating it with a cross-shaped channel offers the advantage of more freely altering the white matter generation region (changing the region to which axons connect) by varying the direction and conditions of electrical stimulation. This enables the simultaneous simulation of axon degradation areas and normal areas within a single BENN sample, allowing for a more accurate representation of a partially damaged brain. This has the effect of enabling the in vitro simulation of the pathological changes associated with progressively developing dementia.

As an example of the present invention, the brain neural network model according to the aforementioned invention can be used for simulating brain neural damage diseases.

The inventors have successfully visualized neurodegeneration and brain compartment-specific degeneration using the brain neural network model according to the present invention. The ability to observe neurodegenerative phenomena in an in vitro model may aid in elucidating the mechanisms of neurodegenerative propagation. Integrating the recently developed three-dimensional microelectrode array (MEA) with this model will enable real-time observation of neural signal changes, potentially contributing to the development of treatments for various neurological disorders.

Furthermore, the present invention, which integrates 3D bioprinting with electrical stimulation, represents a significant advancement in the field of neural tissue engineering and can provide precise neural networks tailored to specific research requirements. In other words, the present invention not only enhances our understanding of neural development and behavior but also provides a robust platform for developing treatments for neurological disorders.

The present invention may be better understood by the following embodiments, which are provided for illustrative purposes and are not intended to limit the scope of protection defined by the appended claims.

### Exemplary Embodiment: Manufacturing of a Brain Neural Network Model

### Manufacturing Example 1: Manufacturing of a Brain Neural Network Model Without Electrodes

First, a frame was fabricated using 3D bioprinting technology on a polypropylene plate, employing biocompatible polyethylene-vinyl acetate (PEVA). The frame was manufactured with a rectangular perimeter, each side measuring approximately 10 mm, and a height of approximately 5 mm (A(1) of FIG. 2). Inlets and outlets were formed on both sides of the frame for subsequent medium injection.

Subsequently, bioink containing neural progenitor cells (NPCs) was printed onto the entire interior of the manufactured frame from above, followed by a cross-linking process at 37° C. for 30 minutes (A(2) of FIG. 2). The bioink containing NPCs is a hybrid bioink combining NPCs with porcine-brain derived decellularized extracellular matrix (P-BdECM) and growth factor-reduced Matrigel (P-BdECM concentration 2 mg/ml, P-BdECM: Matrigel=3:1 mixture).

Furthermore, PF-127 sacrificial material was printed to traverse the center of the hydrogel region within the bioink containing the aforementioned NPC (A(3) of FIG. 2). Specifically, the frame inlet was designated as the origin. The printing nozzle containing the sacrificial material was then immersed into the hydrogel of the bioink, and a printing path was generated within the hydrogel. At this stage, the width of the cell-free region formed was varied by adjusting the size of the nozzle used to print the PF-127 sacrificial material.

Subsequently, the medium was placed inside the frame and stabilized for 30 minutes, after which PF-127 was removed (A(4) of FIG. 2). The medium used was identical to that employed for subsequent cell culture (DMEM F-12, B-27 Supplement, containing Penicillin and Amphotericin-beta).

Subsequently, an acellular bioink was printed into the void space created by the removal of PF-127 (A(5) of FIG. 2). The aforementioned acellular bioink is a hybrid bioink prepared by mixing P-BdECM and Matrigel in a 3:1 ratio to achieve a final ECM concentration of 2 mg/ml. This enables the fabrication of a structure featuring a central acellular region flanked by cell-containing regions on either side, possessing compartments akin to those with separated cell bodies and axons (B of FIG. 2).

Subsequently, the NPCs underwent a maturation process for approximately three weeks to facilitate their neural differentiation. During this maturation process, the fabricated structure may be cultured by introducing media into it. The medium may be introduced onto the frame or injected through an inlet on one side of the frame. Furthermore, during the culture of the structure, differentiation medium may be used, to which approximately 10 ng/ml of brain-derived neurotrophic factor (BDNF) is added.

Upon undergoing this maturation process, the NPCs differentiated into neurons alongside axonal extension, whilst glial cells were also formed. The spatial separation of cellular and acellular regions induced interactions between the two neuronal cell populations above and below, enabling the formation of axonal pathways within the acellular region. This sacrificial material-based fabrication method according to the present invention effectively generated compartmentalized structures. The hydrogel containing cells was cross-linked prior to printing the sacrificial layer, enabling the construction of stable compartmentalized structures.

### Manufacturing Example 2: Manufacturing of a Brain Neural Network Model Including Electrodes

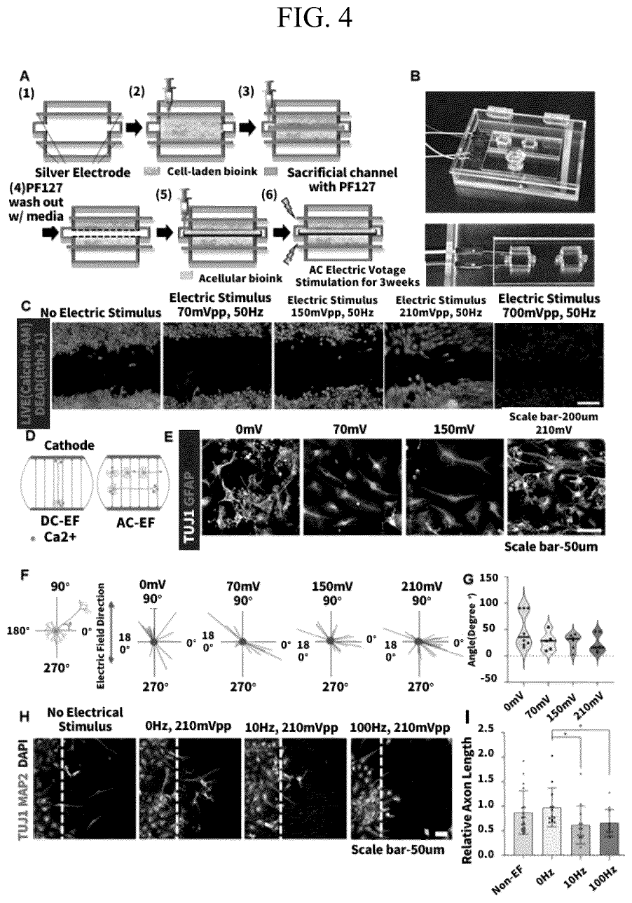

FIG. 4 shows the brain neural network model including electrodes according to an embodiment of the present invention, and the results of electrical stimulation experiments using it ((A) Process of implementing electrical stimulation in the brain neural network model, (B) Acrylic housing for electrical stimulation).

Another crucial feature of the brain is the aligned growth of axons, which functions like an electrical circuit. To simulate this, electrodes were added to the brain neural network model according to the above Example 1 to generate an electric field (A of FIG. 4).

Specifically, silver electrodes were installed lengthwise on both sides of the frame from Example 1. As PEVA is a representative biocompatible material with high electrical conductivity, generating an electric field posed no issues. An additional acrylic housing was fabricated to prevent tensile stress from the electrical cables affecting the framework (B of FIG. 4).

Furthermore, to evaluate the effect of regulating electrical stimulation within the brain neural network model equipped with electrodes, electrical stimulation of varying intensities was applied individually during the approximately three-week maturation process.

Other manufacturing processes were identical to the methods used in the aforementioned Manufacturing Example 1.

### Manufacturing Example 3: Manufacturing of a Brain Neural Network Model Including Electrodes and Cross-Shaped Channels

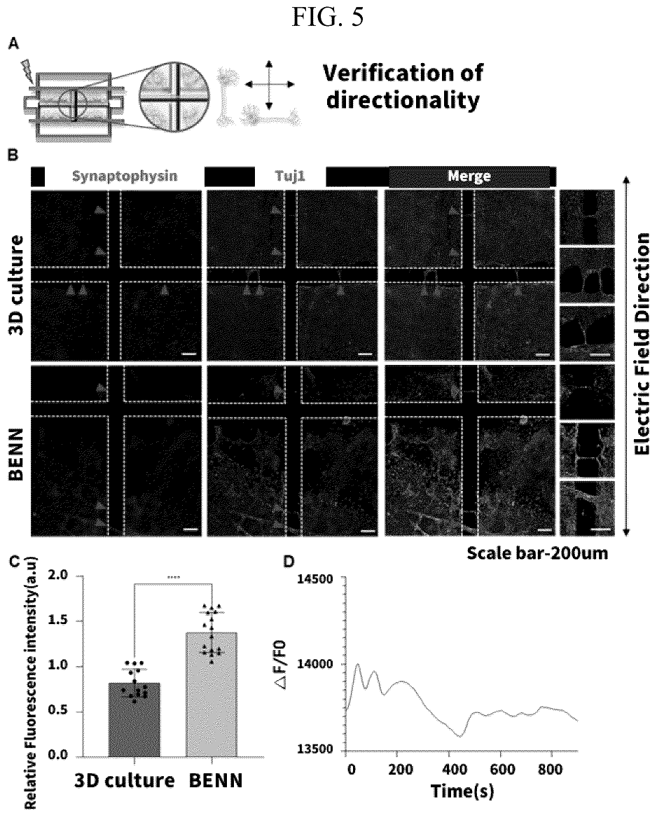

FIG. 5 shows a brain neural network model including electrodes and cross-shaped channels according to an embodiment of the present invention, and the results of electrical stimulation experiments using it ((A) schematic diagram for confirming the direction of axonal growth under an electric field).

Within the brain neural network model comprising electrodes according to an embodiment of the present invention, an acellular region was fabricated using a cross-shaped channel (A of FIG. 5).

In the above manufacturing example 2, printing of the sacrificial material PF-127 to form the acellular region and printing of the acellular bioink were performed in the longitudinal direction and in a direction perpendicular thereto, respectively, to produce a cross-shaped channel where channel 1 and channel 2 intersect.

Otherwise, the remaining manufacturing process was identical to the method used in the above manufacturing example 2.

### Experimental Example 1: Evaluation of Compartmentalization Formation According to the Width of the Cell-Free Zone

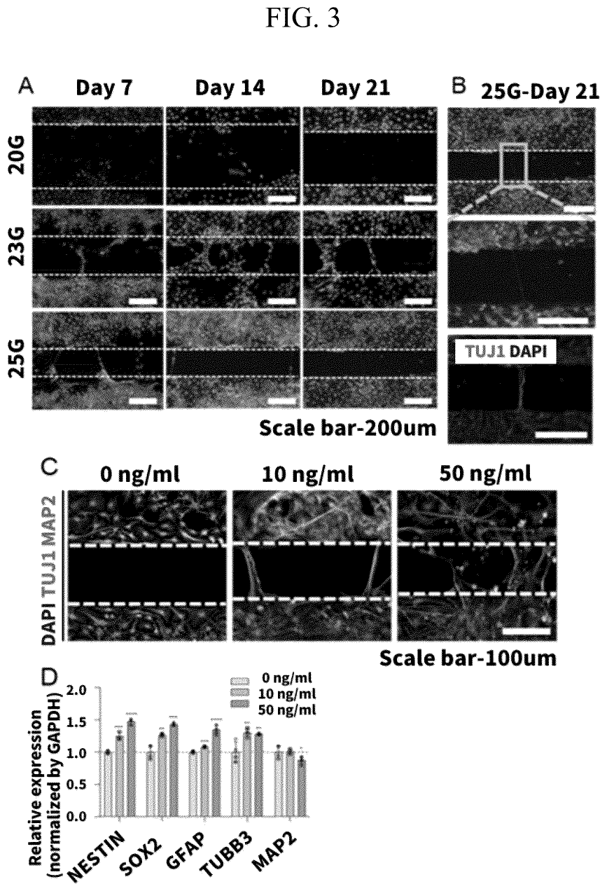

FIG. 3 shows the results of evaluating compartment formation according to the width of the acellular region in the brain neural network model according to an embodiment of the present invention ((A) optimization of the acellular region printing width to induce axonal extension, (B) fluorescence image results of the compartmentalized region after three weeks of maturation).

As axonal pathway formation is significantly influenced by the distance between neuronal populations, the width of the acellular region was varied to evaluate the most suitable width for compartmentalization formation. Specifically, in the above example 1, the width of the acellular region was varied by using nozzles of different sizes for printing the PF-127 sacrificial material and for printing the acellular bioink.

Specifically, using 20 G, 23 G, and 25 G nozzles, we generated acellular zones of 484 μm, 291 μm, and 206 μm width respectively to determine the optimal width for mimicry. After three weeks of maturation, cell migration was observed under the 20 G condition (width 484 μm), but no axonal extension occurred. The migrating cells were confirmed to be inactivated due to insufficient cell-cell interactions. Under 23 G conditions (width 291 μm), both cell and axonal migration were observed, but the migrating cell bodies did not effectively contribute to compartmentalized structure formation. Under 25 G conditions (width 206 μm), axonal extension occurred without cell migration, demonstrating this condition was optimal for compartmentalized structure formation (A of FIG. 3). The experimental results confirmed that the intercellular distance significantly influences both axonal extension and neuronal cell migration, which are essential for neural signal transmission. Specifically, a width of approximately 200 μm was shown to be favorable for axonal pathway formation, enabling inter-regional connectivity while preventing the integration of neuronal cell populations (B of FIG. 3).

### Experimental Example 2: Evaluation of Axonal Extension Promotion via Bdnf Treatment

FIG. 3 shows the results of evaluating the promotion of axonal extension through BDNF treatment in a brain neural network model according to an embodiment of the present invention ((C) immunofluorescence image of the compartmentalized model treated with BDNF, and (D) RT-PCR results for neuronal/glial cell differentiation).

Here, to identify the optimal concentration of brain-derived neurotrophic factor (BDNF) for axonal extension, the expression levels of cell-specific markers were examined following treatment with BDNF at various concentrations. Specifically, in the above example 1, after culturing in medium containing different concentrations of BDNF during the maturation process, the performance of axonal extension promotion by the BDNF was evaluated.

BDNF is known to promote neuronal cell maturation. At 10 ng/ml and 50 ng/ml BDNF treatment, the expression intensity of TuJ1 (class III beta-tubulin) increased significantly, confirming an increase in the axonal cytoskeletal level. Additionally, the degree of tendency for NPC differentiation into neurons to be promoted and differentiation into glial cells to be inhibited was confirmed according to BDNF concentration. The expression intensity of the neuronal marker TuJ1 was similar under 10 ng/ml and 50 ng/ml conditions, whereas the expression intensity of the glial cell marker GFAP was higher under the 50 ng/ml condition (C of FIG. 3). Furthermore, RT-PCR analysis revealed that stem cell markers Nestin and SOX2 were more highly expressed at 50 ng/ml, whereas neuronal markers TUBB3 and MAP2 showed higher expression at 10 ng/ml. Conversely, the glial marker GFAP exhibited greater expression at 50 ng/ml (D of FIG. 3).

In conclusion, for model manufacturing according to the present invention, a 10 ng/ml BDNF treatment was selected as it can promote neuronal differentiation preferentially over glial cell differentiation. Ultimately, the conditions combining sacrificial material printing using a 25 G nozzle with 10 ng/ml BDNF treatment proved most suitable for effectively separating axons and cell bodies, and a brain neural network model was manufactured using these relevant conditions.

### Experimental Example 3: Evaluation of Electrically Stimulated Aligned Axonal Growth Direction

FIG. 4 shows the brain neural network model including electrodes according to an embodiment of the present invention and the results of electrical stimulation experiments using the same: (C) live and dead analysis after electrical stimulation, (D) schematic of calcium ion distribution within neurons under DC and AC electric fields, (E) immunofluorescence images after 14 days to confirm the growth direction of NPCs induced by electric fields under various voltage conditions, (F) analysis of axonal growth pathways for neuronal growth direction, (G) quantitative analysis of axonal growth angles under various electrical stimulation conditions, (H) confirmation via immunofluorescence of NPC growth lengths induced by electric fields under various frequency conditions over 14 days, (I) quantitative numerical quantification of NPC growth lengths).

To evaluate the effect of electrical stimulation control within the brain neural network model equipped with electrodes fabricated according to the above example 2, electrical stimulation of varying ranges was applied during the three-week maturation process. To determine the biocompatible range for neurons, the voltage conditions were adjusted from 0 mVpp to 350 mVpp, and the frequency was varied from 0 Hz to 100 Hz to control axonal growth direction. Among the various voltage conditions, cell survival rates remained similar to the non-stimulated condition at 210 mVpp. However, at voltages exceeding 350 mVpp, some NPCs died, and at 700 mVpp, nearly all NPCs perished (C of FIG. 4). Therefore, a voltage condition of 210 mVpp or below was selected to ensure biocompatibility.

Axonal growth direction is significantly influenced by calcium ion distribution within the neuron. Under a direct current (DC) electric field, calcium ions migrate towards the cathode, leading to axonal growth in that direction and alignment parallel to the electric field vector. Conversely, under an alternating current (AC) electric field, each electrode alternately acts as the cathode, causing calcium ions to concentrate at the midpoint of the electric field. Consequently, axonal growth was anticipated to occur perpendicular to the electric field vector (D of FIG. 4).

Therefore, we investigated whether the expected directionality actually manifests in a brain neural network model equipped with electrodes, and what effect electrical stimulation has on the direction of axonal growth. To prevent the detrimental effects of direct current (such as calcium ion accumulation), we employed alternating current electrical stimulation to generate an aligned axonal network without physical barriers. Applying electrical stimulation with 50 Hz AC for two hours daily over 14 days resulted in neuronal axonal growth being observed perpendicular to the electric field vector. Furthermore, under high AC voltage conditions, axonal growth exhibited even more pronounced alignment, consistently appearing perpendicular to the electric field vector (E of FIG. 4: angle rotated 90 degrees in the image array). To quantitatively evaluate these axonal growth direction results, the direction perpendicular to the electric field was defined as 0 degrees, the direction parallel to the electric field vector was defined as 90 degrees, and the angles were quantified. Consequently, under the 210 mV condition, the primary alignment direction was towards 0°, indicating alignment perpendicular to the electric field, differing from the non-stimulated condition (0 mV) (F and G of FIG. 4: Angle calculations were quantified by defining the direction perpendicular to the electric field (the direction connecting grey matter and white matter) As 0°).

Next, we investigated changes in axons according to frequency conditions. Altering the frequency conditions revealed that axonal growth length was significantly longer at low frequencies, while axons exhibited greater alignment at high frequencies (H and I of FIG. 4). This demonstrates that selecting comprehensive electrical stimulation parameters is crucial for simultaneously regulating both axonal growth length and angle.

### Experimental Example 4: Reproduction of Neural Function in a Brain Neural Network Model Including Cross-Shaped Channels

FIG. 5 shows a brain neural network model including electrodes and cross-shaped channels according to an embodiment of the present invention, and the results of electrical stimulation experiments using this model. ((A) schematic diagram for verifying axonal growth direction under electric field induction (B) immunofluorescence tile scan for verifying axonal network formation (C) quantitative analysis of synaptophysin (D) quantitative analysis of calcium signals for spontaneous neural activity in the brain neural network).

Using the electrode and brain neural network model including a cross-shaped channel manufactured according to the above example 3, we attempted to regulate axonal directionality under electrical stimulation after adding the cross-shaped channel within the brain neural network (A of FIG. 5).

In the brain neural network without applied AC electrical stimulation, axonal connections were observed to extend in all directions within the channel. Conversely, in the brain neural network subjected to AC electrical stimulation, it was confirmed that axonal connections aligned only in the direction perpendicular to the electric field (B of FIG. 5). These results demonstrate that AC electrical stimulation effectively controls axonal directionality and can guide axonal growth perpendicular to the electric field. This ability to control the direction of axonal growth holds significant implications for the development of structured neural networks.

Subsequently, within the brain neural network subjected to AC electrical stimulation, neural maturity was assessed using synaptophysin—a functional synaptic marker expressed in mature neurons—and calcium imaging to analyse neural activity. Within the brain neural network subjected to AC electrical stimulation, compared to the model not receiving AC electrical stimulation, the degree of synaptophysin expression increased (C of FIG. 5), and spontaneous calcium influx was observed (D of FIG. 5). This indicates that within the brain neural network subjected to AC electrical stimulation, neurons mature and functional neural activity proceeds. This spontaneous calcium activity highlights the neurons' capacity to participate in intrinsic electrical signal transmission, a hallmark of mature neural function. Consequently, the brain neural network subjected to AC electrical stimulation not only induces axonal growth but also significantly enhances the maturation and functional integration of the neural network.

### Experimental Example 5: Application of the Brain Neural Network Model to an Alcohol-Induced Neurodegenerative Model

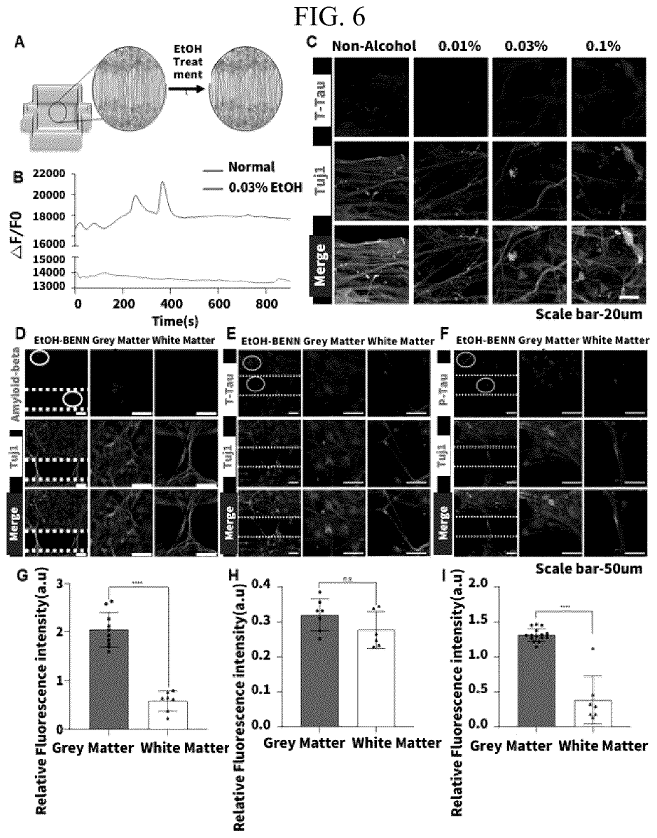

FIG. 6 shows the results of applying the brain neural network model according to an embodiment of the present invention to an alcohol-induced neurodegenerative model. (A) schematic diagram of the application of the brain neural network model to an EtOH-induced neurodegenerative model (B) acute calcium signaling dysfunction under EtOH treatment (C) axonal deformation due to chronic alcohol consumption (D) immunofluorescence results for amyloid-beta (E) immunofluorescence results for t-tau (F) immunofluorescence results for p-tau (G) quantitative analysis of immunofluorescence intensity for amyloid-beta, (H) t-tau, (I) p-tau).

The pattern of neurodegeneration varies slightly across different brain regions. When neurodegeneration occurs, synaptic activity in neurons declines, axonal deformations such as curvature and swelling develop, and neurofibrillary tangles (NFTs) primarily form in the hippocampus and entorhinal cortex. This is associated with a rapid reduction in grey matter volume in these regions at the onset of the disease. Thus, understanding the regional specificity of neurodegeneration in vitro is crucial for elucidating the underlying mechanisms of brain degeneration.

Therefore, as an application of the brain neural network model, alcohol-induced neurodegeneration was simulated in vitro. The alcohol concentration was selected to reflect blood alcohol concentration (BAC), with 0.1% corresponding to 0.1 g of alcohol in 100 ml of medium. The brain neural network model prepared according to Example 1 of the present invention was directly treated with a 0.03% (0.03 g/100 ml) EtOH mixed medium. This concentration corresponds to the social alcohol consumption conditions (conditions when an adult male consumes half a bottle of soju in a short period) confirmed through existing in vitro experiments. In the acute model, neuronal activity was assessed one hour after alcohol treatment; in the chronic model, EtOH-mixed medium was administered daily for three weeks (A of FIG. 6).

To evaluate acute model function, neuronal signaling activity was assessed via calcium imaging in a brain neural network model one hour after EtOH treatment. The normal model (without EtOH treatment) exhibited active signaling activity, including spontaneous calcium responses, whereas the alcohol-treated model showed inhibited signaling activity (B of FIG. 6). This indicates that acute alcohol exposure alone can significantly reduce neuronal activity.

Next, the degree of axonal deformation was assessed in the grey and white matter compartments of the brain neural network model. No significant axonal deformation was observed under normal conditions or low alcohol treatment (0.01% EtOH). However, at 0.03% (social alcohol consumption) and 0.1% (hazardous alcohol treatment) EtOH concentrations, axons in the BENN's white matter exhibited a curved rather than straight form, indicating the occurrence of neurodegenerative phenomena (C of FIG. 6).

To assess the degree of neurodegeneration in grey and white matter regions of the brain neural network model, we investigated amyloid-beta accumulation, total tau (t-tau), and phosphorylated tau (p-tau) formation. Amyloid-beta accumulation, a toxic byproduct of neurodegeneration, was higher in grey matter regions than in white matter regions (D and G of FIG. 6). Formation of t-tau, which normally stabilizes axonal structures but appears abnormally in neurodegenerative states, showed no significant difference between grey and white matter (E and H of FIG. 6). Another neurodegenerative byproduct, phosphorylated tau (p-tau), was found to be higher in grey matter regions than in white matter regions (F and I of FIG. 6).

These results demonstrate that the constructed brain neural network model possesses the potential to visualize the mechanisms of region-specific neurodegeneration in vitro, highlighting the utility of brain neural network models in studying neurodegenerative processes.

The foregoing description has illustrated and explained the present invention in relation to specific, preferred exemplary embodiments. However, it will be apparent to those skilled in the art that the present invention may be variously modified and altered without departing from the technical features or scope defined by the appended claims.

1. A manufacturing method of brain neural network model, the method comprising: printing a first bioink containing neural progenitor cells (NPCs) within a frame having a predetermined shape; printing a sacrificial material onto a portion of the printed first bioink; removing the printed sacrificial material; printing a second bioink, which is devoid of cells, onto the area where the sacrificial material has been removed; and maturing the printed first bioink and second bioink.

2. The manufacturing method of claim 1, wherein the frame has a shape having a bottom portion, a side perimeter portion, and an upper opening.

3. The manufacturing method of claim 1, wherein the first bioink is formed by including neural progenitor cells (NPCs), brain decellularized extracellular matrix (BdECM), and Matrigel.

4. The manufacturing method of claim 1, wherein the printing the sacrificial material is performed transversely across the longitudinal direction of the printed first bioink.

5. The manufacturing method of claim 1, wherein the printing the sacrificial material involves discharging the sacrificial material into the printed first bioink bioink while the nozzle discharging the sacrificial material is in a locked state.

6. The manufacturing method of claim 1, wherein the removing the sacrificial material involves placing the medium inside the frame and leaving it to remove the sacrificial material.

7. The manufacturing method of claim 1, wherein the second bioink is formed by including brain decellularized extracellular matrix (BdECM) and Matrigel.

8. The manufacturing method of claim 1, wherein the maturation process involves placing medium within the frame containing the printed first bioink and second bioink, and culturing the neural progenitor cells (NPCs) from the first bioink.

9. The manufacturing method of claim 1, wherein the maturation process involves placing a medium containing brain-derived neurotrophic factor (BDNF) within the frame containing the printed first bioink and second bioink, and culturing the neural progenitor cells (NPCs) of the first bioink for a period ranging from two to four weeks.

10. The manufacturing method of claim 9, wherein the medium containing brain-derived neurotrophic factor (BDNF) is a medium containing brain-derived neurotrophic factor (BDNF) at a concentration of 5 ng/ml to 15 ng/ml.

11. A brain neural network model manufactured according to the manufacturing of method of claim 1, wherein the brain neural network model includes a first region formed by printing a first bioink containing neural progenitor cells (NPCs), and a second region formed by printing a second bioink without cells onto a portion of the printed first bioink.

12. A brain neural network model comprising a first region formed by printing a first bioink containing neural progenitor cells (NPCs) within a frame of predetermined shape; and a second region, formed by printing a cell-free second bioink, which is an area where a sacrificial material is printed onto and subsequently removed from a portion of the printed first bioink, wherein the neural progenitor cells (NPCs) in the first region differentiate into neural cells, and axons extend into the second region.

13. The brain neural network model of claim 12, wherein the second region is formed in the longitudinal direction of the first region.

14. The brain neural network model of claim 13, wherein the second region has a width within the range of 100 μm to 300 μm.

15. A manufacturing method of the brain neural network model of claim 12, wherein a medium containing brain-derived neurotrophic factor (BDNF) at a concentration of 5 ng/ml to 15 ng/ml is further included within the frame.

16. The brain neural network model according to claim 12, further comprising an electrode for applying an electric field to the first and second regions; wherein the neural progenitor cells (NPCs) in the first region differentiate into nerve cells, and axons extend into the second region.

17. The brain neural network model of claim 16, wherein the second region is formed in the longitudinal direction of the first region, and the electrode is positioned in a direction perpendicular to the second region.

18. The brain neural network model according to claim 12, further comprising a third region formed by printing a third bioink, which is cell-free, after printing and removing a sacrificial material on a portion of the printed first bioink and second bioink, wherein the second region and the third region have an intersection point, and wherein the neural progenitor cells (NPCs) in the first region differentiate into neural cells, and axons extend into one or more of the second and third regions.

19. The brain neural network model according to claim 12, wherein the brain neural network model is a brain neural damage disease model.
